(19) United States
(12) Patent Application Publication
(10) Pub. No.: US 2026/0060626 A1
(43) Pub. Date: Mar. 5, 2026

(54) COMPUTER-IMPLEMENTED METHOD FOR OPERATING AN IMAGE PROCESSING DEVICE AND IMAGE PROCESSING DEVICE

(71) Applicant: Siemens Healthineers AG, Forchheim (DE)

(72) Inventors: Magdalena HERBST, Erlangen (DE); Ramyar BINIAZAN, Nuernberg (DE); Johannes VOIGT, Bayreuth (DE); Ludwig RITSCHL, Buttenheim (DE)

(73) Assignee: Siemens Healthineers AG, Forchheim (DE)

(21) Appl. No.: 19/311,003

(22) Filed: Aug. 27, 2025

(30) Foreign Application Priority Data
Aug. 29, 2024 (DE) 10 2024 208 223.6

(51) Int. Cl. A61B 6/46; G06V 10/764; G06V 20/70; G16H 30/20; G16H 30/40

(52) U.S. Cl. CPC A61B 6/468; A61B 6/461; G06V 10/764; G06V 20/70; G16H 30/20; G16H 30/40; G06V 2201/07

(57) ABSTRACT

One or more example embodiments relates to a computer-implemented method for operating an image processing device with a display device, an input device and a controller, the method comprising for processing an X-ray image for display on the display device, applying at least one orienting function which adjusts a display orientation for the display of the X-ray image to the X-ray image by the controller; establishing at least one orientation specification information item based on a user input made via the input device; and selecting the display orientation using the orienting function and the orientation specification information item.

### CROSS-REFERENCE TO RELATED APPLICATION(S)

The present application claims priority under 35 U.S.C. § 119 to Germany Patent Application No. 10 2024 208 223.6, filed Aug. 29, 2024, the entire contents of which is incorporated herein by reference.

### FIELD

One or more example embodiments relates to a computer-implemented method for operating an image processing facility with a display facility for displaying an X-ray image, an input facility and a control facility, wherein for processing an X-ray image for display on the display facility, at least one orienting function which adjusts a display orientation for the display of the X-ray image is applied to the X-ray image by the control facility. In addition, the invention relates to an image processing facility, a computer program and an electronically readable data carrier.

### RELATED ART

Radiographic X-ray images of examination objects, in particular patients, that are recorded with medical X-ray facilities are typically passed on for diagnostic evaluation by a diagnosing person. It can, however, often occur that the orientation of the X-ray image is less suitable for the diagnosing person to undertake a diagnosis, so that it is known that users rotate displayed X-ray images manually into an orientation that seems suitable to them. This applies also to other adjustments, for example the removal of a collimator shadow and/or an annotation. The specifically desired adjustments, in particular the display orientation, can however be subjective for some body parts and/or extremities. For example, radiography X-ray images of a knee in a lateral view can be adapted in their orientation on the basis of the aim of a straight femur or a straight tibia.

It has previously been proposed in the prior art initially to prepare the X-ray image before the display, for example at a diagnostic workstation, with regard to the orientation. Therein, a display orientation is proposed, for example for different application cases, according to particular radiographic instructional materials. However, practical use has shown that users still adapt the result of the orienting function such that different ultimate results come about.

### SUMMARY

The manually made adaptations result in a high level of effort so that the user cannot always concentrate completely on the diagnostic task and/or other processing task. In addition, the manual adaptations lead to different results for display and also for saving, in particular following annotation, not only with regard to the orientation of the X-ray image, the comparability of which is sometimes made more difficult.

One or more example embodiments enables an improvement in the diagnostic capacity of X-ray images and/or the comparability of evaluated X-ray images.

In accordance with example embodiments, a computer-implemented method, an image processing facility, a computer program and an electronically readable data carrier are provided as claimed in the independent claims enable an improvement in the diagnostic capacity of X-ray images and/or the comparability of evaluated X-ray images. Advantageous developments are disclosed in the subclaims.

### BRIEF DESCRIPTION OF THE DRAWINGS

Advantages and details are disclosed in the exemplary embodiments described below and by reference to the drawings. In the drawings:

FIG. 1 shows a flow diagram of an exemplary embodiment,

FIG. 2 shows a possible output on a display facility for selecting an offset angle,

FIG. 3 shows a possible output on the display facility for selecting an anatomical landmark,

FIG. 4 shows a possible output on the display facility for placing annotations,

FIG. 5 shows a possible output on the display facility for selecting a region of a collimator shadow that is to be removed, and

FIG. 6 shows schematically an image processing facility according to one or more example embodiments.

### DETAILED DESCRIPTION

In a method of the aforementioned type, it is provided according to one or more example embodiments that, from a user input made via the input facility, at least one orientation specification information item is established and the display orientation is selected by way of the orienting function making use of the orientation specification information item.

The orientation specification information item is therefore provided as input data of the orienting function. Thus the orienting function can initially establish the display orientation taking account of the orientation specification information item and then adjust it. The adjustment to the display orientation should herein be understood to mean that if the image orientation already corresponds to the display orientation, this is maintained and if the image orientation does not correspond to the display orientation, the X-ray image is rotated into the display orientation. Orienting functions and/or the functionality provided thereby are therefore also known as “auto-rotate”.

According to one or more example embodiments, it is therefore proposed, even before the processing of individual X-ray images, in particular radiography X-ray images, to permit a modification with regard to the display orientation by way of a user input, wherein the established orientation specification information item can be, and preferably also is, used for a plurality of X-ray images. For example, it can therefore be provided that X-ray images of at least one patient cohort are processed with the same orientation specification information item. In this way, therefore, not only can a reduction in the effort for the diagnosis itself be achieved, since a complex manual adjustment of the display orientation can be dispensed with, but the possibility of standardization of the display orientation for different patients, in particular whole cohorts and/or hospitals/radiology practices and/or across all of them, can also be brought about. In other words, a clinical standardization can be achieved that is not only suitable for the comparison of X-ray images from different patients, but that also, in general, is conducive to the enhancement of diagnostic quality since the recognition of findings is simplified by the standardization.

The procedure described therefore permits, in particular, a display orientation and/or the form of its establishment to be specified for a complete medical facility, for example a hospital, a radiology practice or suchlike, in order also to bring about a type of standardization, including for different users, in particular diagnosing persons. It can therefore be provided that the orientation specification information item is firmly specified as a standard for at least one user group, in particular for users working together in a hospital and/or a radiology practice.

In one or more example embodiments, it can be provided that orientation specification information is established for a plurality of X-ray image classes, wherein the X-ray image is classified into an X-ray image class before the application of the orienting function, and the orienting function uses the orientation specification information item that is associated with the classification result. X-ray image classes can be defined according to particular uses and/or recording programs, for example ribcage/lung lateral, ribcage/lung anterior-posterior, knee lateral, hand anterior-posterior and suchlike. A classification function for automatic classification of an X-ray image can operate on the image data of the X-ray image, for example as a trained classification function, and/or can use metainformation regarding the X-ray image, for example recording parameters and/or background information, as can be contained, for example, in a patient file, an information system and/or as DICOM-metadata in an X-ray image. A user can therefore, for example, adapt the output of the orienting function for each body part and/or each viewing direction (projection direction), so that a highly adaptable preset results.

One or more example embodiments can provide that in the orienting function, in particular by way of a subfunction trained via machine learning and/or on the basis of a guideline information item and/or on the basis of an anatomical landmark in the X-ray image, a preliminary orientation is established by way of evaluating the X-ray image and the orientation specification information item includes an offset angle which is added to establish the display orientation of the preliminary orientation. Investigations have revealed that, starting from the result of conventional orienting functions, in particular auto-rotate functions, users often rotate by similar angles in order to adjust their preferred and/or particularly suitable orientation of the X-ray image. Therefore, in preferred exemplary embodiments, it may be advantageous to map this and to provide the orientation specification information item comprising an offset angle relative to a preliminary orientation. Therein, the preliminary orientation can be provided, for example, on the basis of guidelines, thus for example in a rule-based manner, and/or can result from the shape of an anatomical feature, for example a bone, which, in particular, can be determined by way of the evaluation of the X-ray image. It is, however, possible to achieve an X-ray image-specific establishment by way of a trained subfunction in that preliminary orientations are provided as a ground truth and that training takes place by way of machine learning, wherein the ground truth can also be derived from guidelines, but also from user feedback and suchlike. The trained subfunction can then use the X-ray image as input data and outputs a corresponding preliminary orientation. Thereon, the offset angle is then applied as a type of delta rotation.

A development that is particularly advantageous in this context provides that for receiving user input, an operating element that is rotatable for adjusting the offset angle having a pictogram that shows schematically an X-ray image of an X-ray image class for which the offset angle is to be selected, is used, wherein the starting orientation of the pictogram is selected according to a predicted orientation without using the orientation specification information item. Herein, it can be provided that the predicted display orientation is selected according to a guideline and/or a statistic and/or another specification.

The predicted orientation can therefore correspond to the preliminary orientation, in particular if it is established as specific to an X-ray image class, particularly advantageously using the orienting function and/or its corresponding subfunction. An X-ray image class-specific preliminary orientation can be established, as previously mentioned, in particular from guidelines and/or user feedback messages. If machine learning is used, it is also conceivable that as the predicted orientation, a mean value of the ground truth of the training data for the respective X-ray image class is used.

The user can thus rotate the operating element displayed on the display facility and, with the aid of the pictogram, receives a visual schematic feedback of the impression arising, so that he can select the orientation suitable for him or a user group extremely intuitively. Therein, the pictogram is generated in the orientation of a “ground truth”, specifically as described, preferably according to the preliminary orientation, so that therefore the adaptation can be performed specifically in respect of the offset angle. The rotation can herein be achieved via the input facility. In general, it is the case that the input facility can also be integrated at least partially into the display facility, for example that the display facility can comprise a touch screen which also serves as an input means of the input facility.

In the context of the rotatable operating elements, it can also be suitable if, on provision of user inputs for a plurality of X-ray image classes, a plurality of operating elements is provided in a representation, for example arranged in a row adjoining one another and/or in a matrix-like manner. On the basis of the schematic pictogram, the operating elements can be distinguishable and clearly assignable.

In a suitable embodiment of the present invention, it can be provided that for receiving the user input, a selection operating element is used which has a selection image which shows schematically anatomical landmarks of an X-ray image of an X-ray image class for which the orientation specification information item is to be selected, wherein a landmark picked out via the selection operating element in the orientation specification information item is stored as specifying the display orientation. Such a specification of the landmark can be used additionally or alternatively to the offset angle, since it is conceivable, for example, to deviate again from a fundamental preliminary orientation in which a picked-out landmark is arranged, for example horizontally or vertically in the image, by an offset angle. However, it is naturally also conceivable to specify just the picked-out landmark as the orientation specification information item.

Thus, the possibility can be given to the user to pick out at least one anatomical landmark in relation to which the X-ray image is to be rotated. For example, the user can choose for the knee that the tibia is always displayed vertically, that the wrist is always arranged at the bottom and suchlike. Herein also, the possibility is given to the user to make a selection intuitively in that a selection operating element is provided in which the corresponding anatomical landmarks are shown able to be picked out in the selection image. In a suitable development, it can be provided that with the selecting of an anatomical landmark, the selection image is adapted to display a resultant orientation. Thus, before a final selection, different anatomical landmarks are activated in order to obtain a preview. It is conceivable in this context also to use further operating elements in order to be able to adjust the manner of the relation of the orientation to an activated or picked-out anatomical landmark.

Particularly advantageously, apart from the orientation specification information item, further specification information can also be preset.

It is thus conceivable, in preferred exemplary embodiments, that from the user input, an annotation specification information item is also established, in particular for each X-ray image class, wherein for the processing of the X-ray image for display after the application of the orienting function, the X-ray image is annotated in an enhancement function making use of the annotation specification information item. Annotations which can already be added automatically to an X-ray image in the context of the processing by way of the control facility comprise for example recording parameters, information regarding the view (lateral, anterior-posterior and suchlike), information regarding recording circumstances (for example, position of the patient during the recording (standing, sitting, lying, . . . ), difficult recording if the patient cannot assume particular positions, and suchlike) and information regarding the anatomy being represented, for example, in the case of an extremity, whether it is on the right side or the left side. Directions, in particular as anatomical direction information, can also be added automatically as annotations. Annotation information that is to be represented can be established, for example, by way of evaluating the X-ray image itself, but also from metainformation relating to the X-ray image. For example, recording parameters and/or background information, as can be contained, for example, in a patient file, an information system and/or as DICOM-metadata in an X-ray image, can be used.

Suitably, the annotation specification information item can relate to the arrangement of an annotation of particular content in the X-ray image, in particular relative to at least one anatomical landmark. Therefore, the specification of a particular arrangement of at least one annotation of particular content is initially entirely possible, for example, in principle, in a left or right top corner. A relative specification regarding an anatomical landmark is also possible, for example, if it is known where a free space is available and/or where, for a diagnostic evaluation, less or non-relevant anatomy is present.

In a specific embodiment in this context, it can be provided that for establishing the portion of the user input relating to the annotation specification information item, an input region is displayed on the display facility, wherein the input region comprises a schematic map of an X-ray image of the X-ray image class for which the annotation specification information item is to be selected, in particular in the predicted orientation, and displaceable annotation elements which can be displaced to positions within the display image in order to input the portion of the user input. Markers and/or text and/or image content of the annotations can therefore be freely placed in the schematic map, possibly in different modes absolutely and relative to anatomical landmarks. Preferably, the schematic map also shows schematically at least one anatomical landmark in relation to which the arrangement can be defined. In particular, the schematic map is displayed centrally and the different annotations are placed on at least two sides about the schematic map such that they can be moved, for example via a cursor and/or in the case of a touch screen also via a finger, to their desired target position within the schematic map, in order to input the portion of the user input relating to the annotation specification information item. If the orientation specification information item has already been established, the predicted orientation can be updated and/or established taking account of it, so that particularly advantageously, it is possible to proceed from what is desired with regard to the orientation.

In preferred developments of the method, it can further be provided that from the user input, a cropping specification information item is also established, in particular for each X-ray image class, wherein for the processing of the X-ray image for display, the X-ray image is trimmed in a trimming function making use of the cropping specification information item regarding a collimator shadow in the X-ray image. Even with regard to the treatment of the collimator shadow, different specifications and/or practices are conceivable since conclusions can also still be drawn, in part, from the collimator shadow and at least some users and/or user groups would like to use them. The cropping in respect of the collimator shadow can also be partially subject, in particular, for certain user groups, to rules that can be mapped via a pre-setting via the cropping specification information item, in particular in order to achieve a standardization.

In specific terms, it can be provided in this context that in order to establish the portion of the user input relating to the cropping specification information item, in particular supported by a visual representation, different removable regions of the collimator shadow, in particular comprising complete removal and/or no removal and/or half removal, are offered for selection via the input facility and/or a sectioning element that can be manipulated by the user for defining removable regions is used. For example, it is again conceivable to use a, or the, schematic map of the X-ray image, around which, again schematically, removable regions of the collimator shadow are shown. The regions can then be picked out, for example by way of a direct interaction therewith, although it is also possible to make the selection by other means. The regions comprise, in particular, a complete removal of the collimator shadow, a half removal of the collimator shadow and/or no removal of the collimator shadow at all. Furthermore, a sectioning element can also be offered to the user for definition by the user of regions that can be removed. For example, it is conceivable, as a starting position, to divide the collimator shadow centrally by way of the sectioning element comprising suitable lines. The line can be displaced by the user so that it does not lie exactly in the middle, but rather at a freely selectable site between complete and none at all. It is particularly preferred to combine both embodiments as the marking for “half removal” as the sectioning element.

It should also be noted at this point that the embodiments relating to X-ray image classes, user groups and for standardizing naturally continue to apply accordingly for the annotation specification information item and/or the cropping specification information item in a similar manner. This means that the annotation specification information item and/or the cropping specification information item can also be firmly specified as a standard for the at least one user group and/or annotation specification information and/or cropping specification information can be established for the majority of X-ray image classes. Finally, it is naturally particularly advantageous if X-ray images of at least one patient cohort are processed with the same annotation specification information and/or cropping specification information, in order also in this regard to allow an outstanding evaluation capability, comparability and, in particular, clinical standardizing capability.

In addition to the method, one or more example embodiments also relates to an image processing facility with a display facility for displaying an X-ray image, an input facility for receiving a user input and a control facility, wherein for processing an X-ray image for display on the display facility, the control facility has:
an establishing unit for establishing an orientation specification information item from a user input effected via the input facility, and
an orienting unit for applying to the X-ray image an orienting function which adjusts a display orientation for the X-ray image for display, wherein the display orientation is selected by way of the orienting function making use of the orientation specification information item.

In other words, the control facility is configured for carrying out the method according to one or more example embodiments. All the embodiments of the method can be transferred similarly to the image processing facility according to one or more example embodiments and vice versa, so that the advantages mentioned above can therefore also be obtained with the image processing facility.

The control facility (also referred to as a controller) has, in particular, at least one processor and at least one storage means. By way of hardware and/or software, functional units can be formed in order to perform steps of the method according to the one or more example embodiments. Apart from the aforementioned establishing unit and the aforementioned orienting unit, further functional units are naturally also conceivable, for example in order to enable advantageous developments of the method according to the one or more example embodiments. Thus, the control facility can in particular also have a display unit in order to represent the X-ray image on the display facility following the processing.

In some exemplary embodiments, the image processing facility can be part of an X-ray facility with which the X-ray images that are to be processed are recorded. However, it is equally conceivable to realize the image processing facility completely independently of the actual X-ray facility that records X-ray images. In particular, the image processing facility is then also independent in its function from the actual X-ray facility and can thus suitably process and display X-ray images from different X-ray facilities, in particular in a standardized manner. In other words, the image processing facility can be configured to be independent of the scanner.

A computer program according to one or more example embodiments is able to be loaded directly into a storage means of a control facility of an image processing facility and has program means such that when the computer program is executed on the control facility, said control facility is caused to carry out the steps of a method according to one or more example embodiments. The computer program can be stored on an electronically readable data carrier according to one or more example embodiments which therefore has control information stored thereon, which comprises at least a computer program according to one or more example embodiments and is configured such that, on use of the data carrier in a control facility of an image processing facility, said control facility is configured to carry out a method according to one or more example embodiments. The data carrier is preferably non-transient, for example a CD-ROM.

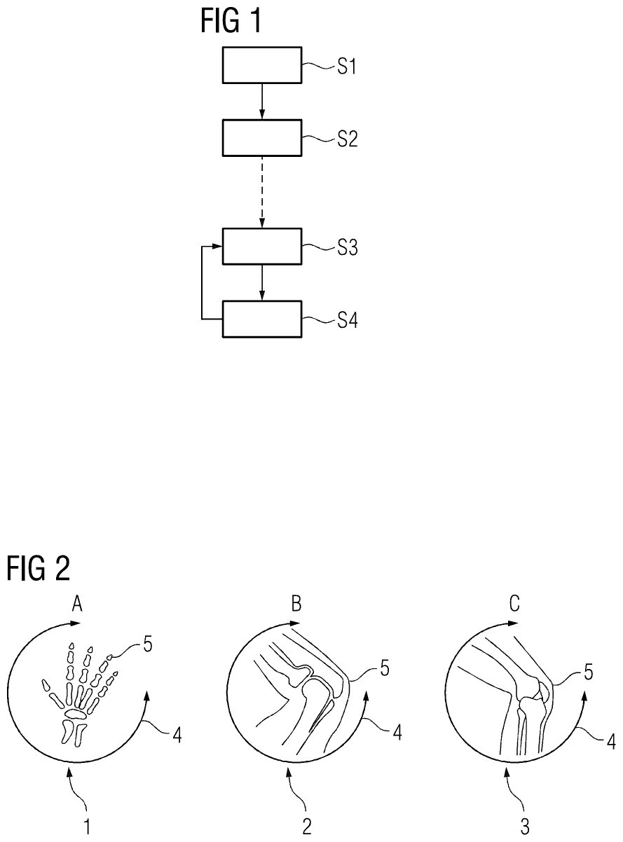

FIG. 1 shows a flow diagram of an exemplary embodiment, as can be carried out by way of a control facility of an image processing facility, which also has a display facility and an input facility. The image processing facility can be part of a picture archiving and communication system (PACS) and/or can be connected to one such, in particular in order to permit access to X-ray images that are to be processed and displayed, and have been recorded with at least one medical X-ray facility.

Therein, firstly in a first section of the method shown, steps S1 and S2, presettings are made that are to be applied subsequently to a plurality of X-ray images, in particular for at least one user group. For this purpose, a user interface with different views is output in step S1 on the display facility and is used for receiving a user input via the input facility. In a step S2, from the user input, different specification information items are established which are used, in a second, later section of the method shown, for processing X-ray images in a step S3 before their display for diagnosis in a step S4. The steps S1 and S2 can therein also be interleaved with one another, for example following each view of the user interface, an associated specification information item can be established before the next view is displayed for the next type of specification information. In the present case, orientation specification information items, annotation specification information items and cropping specification information items are established, specifically for different correspondingly user-selectable X-ray image classes that correspond to different uses and types and contents of X-ray images associated therewith.

In the present case, the specification information items, that is the orientation specification information items, the annotation specification items, and the cropping specification information items, are defined in advance for at least one user group as standard in the first section, for example for at least one medical facility such as a hospital and/or a radiological practice. In this way, the specification information items are used in the following for the processing of X-ray images from different patients, in particular a plurality of patient cohorts.

FIGS. 2 to 5 illustrate different views of the user interface for the reception of portions of the user input, respectively for particular specification information items.

Thus, FIG. 2 shows, in the present case for each of three different X-ray image classes that are identified in FIG. 2 as A, B and C, an operating element 1, 2, 3 which is configured to be rotatable via a suitable input means of the input facility, as is also indicated by the arrows 4. Each operating element 1, 2, 3 also comprises a pictogram 5, which shows schematically an X-ray image of the respective X-ray image class. Since the X-ray image class A concerns, in the present case “Hand, anterior-posterior”, the X-ray image class B “Elbow, lateral”, and the X-ray image class C “Knee, lateral”, corresponding schematic pictograms 5 are shown. These X-ray image classes A, B, C and pictograms 5 are naturally to be understood as purely exemplary. The view of the user interface can also be generated such that a suitable instruction text is also displayed, for example “In which orientation are the X-ray images to be displayed? Please adapt them for each application.”

The orientation of the pictograms 5 therein corresponds, at least substantially, to a predicted orientation which, in the present case, corresponds to a preliminary orientation which is established in step S3 by way of an orienting function before an offset angle to be specified by the operating elements 1, 2, 3 is applied. The predicted orientation and thus the preliminary orientation can be established, for example, on the basis of a guideline information item and/or the course of an anatomical landmark. If, for establishing the preliminary orientation in an orienting function, a trained subfunction is used, for example, the predicted orientation can be picked out as a mean value of the ground truths used for its training. It has proved to be the case that often only very slight deviations occur, so that for symbolic intuitive user information, an estimate of this type is sufficient for an abstract pictogram 5. When proceeding from guidelines and/or anatomical landmarks, the predicted orientation corresponds, in particular, exactly to the preliminary orientation for an X-ray image of the X-ray image class. In the case of the anatomical landmarks, the course of the anatomical landmark in the schematic pictogram is also known (and ideally also visible).

A user can now rotate the operating elements 1, 2, 3 with the pictograms 5 in order to adjust the aforementioned offset angle. If the rotated operating elements 1, 2, 3 and thus the pictograms 5 deviate from the predicted orientation, in step S2, the offset angle as the orientation specification information item, or otherwise 0°, is established as the deviation.

Herein, a large offset angle can also be set, for example starting from “hand pointing up” in the case of the exemplary X-ray image class A, to “hand pointing down”, so that the offset angle would be 180°. Small offset angles, for example in the range from −15° to +15°, are also possible.

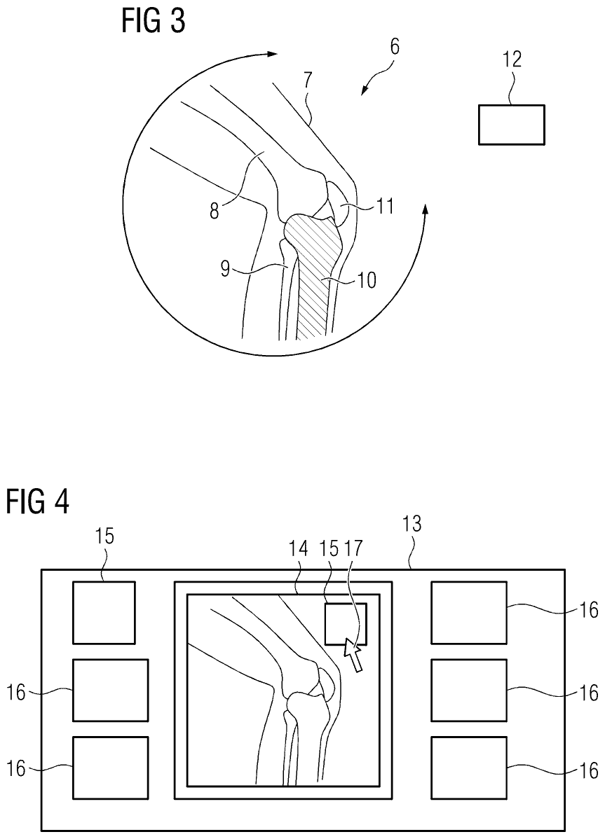

FIG. 3 shows an alternatively or additionally usable view of the user interface for establishing the portion of the user input that relates to the orientation specification information item. Therein, in the present case for a single X-ray image class, for example “knee, lateral”, a selection operating element 6 having a selection image 7 is shown which shows schematically anatomical landmarks 8, 9, 10, 11 of an X-ray image of the X-ray image class. The anatomical landmarks herein comprise, by way of example, thigh bone, lower leg bones and a kneecap. The anatomical landmark 10 is therein selected. Since the selection of this anatomical landmark 10 would mean that the X-ray image would be oriented such that the anatomical landmark 10 extends vertically, with the selecting of the anatomical landmark 10 the selection image 7 would be rotated such that it is also visible in the selection operating element 6. By way of a confirmation area 12, the choice made can then be confirmed as the selection, so that the portion of the user input comes into being. In step S2, the landmark 8, 9, 10, 11 thus picked out via the selection operating element 6 is stored in the orientation specification information item as specifying the display orientation.

Here also, in the view, an explanatory text can additionally be output, for example “Along which anatomical structure should the orientation occur? Please mark it in the image.”

If both views, that is that of FIG. 3 and that of FIG. 2, are used, it is suitable initially to use the view of FIG. 3, since the picked-out anatomical landmark 8, 9, 10, 11 then specifies the preliminary and predicted orientation which still has to be adjusted by the offset angle.

FIG. 4 shows a possible third view of the user interface that can be used in order to establish a portion of the user input relating to the annotation specification information item. Therein, an input region 13 is displayed on the display facility, which comprises a schematic map 14 of an X-ray image of the X-ray image class and displaceable annotation elements 15, 16. The map 14 is itself shown in the predicted orientation, although if the orientation specification information item for the X-ray image class is already available, said orientation can be updated to take account of it, and therefore can take account of the picked-out landmarks and/or the offset angle. The annotation element 15 which displays, in the present case, whether the extremity shown is a right or a left extremity, in this case a right or a left knee, has already been displaced with an input means, in this case a cursor 17, to a position within the map 14. Therein, this position in step S2 can be stored in the annotation specification information item, in particular dependent upon the input mode, as an absolute target position at which the annotation in the X-ray image is to be enhanced or as a target position relative to at least one anatomical landmark 8, 9, 10, 11 at which the annotation in the X-ray image is to be replaced. It is possible to change between the input modes, for example via a button (not shown).

Finally, FIG. 5 shows a view of the user interface for selecting a portion of the collimator shadow 18 that is to be removed, and therefore for receiving a portion of the user input relating to the cropping specification information item. The virtual representation shown there for assistance during the selection again comprises the map 14, in this case with its collimator shadow 18 indicated, it being subdivided by dashed, in particular, colored lines 19 such that different removable regions of the collimator shadow 18 are displayed in order to assist the selection. In the present case, the collimator shadow 18 can be removed completely, by half (that is up to half, starting from the current image region) or not at all. In exemplary embodiments, however, the central colored line 19 can also serve as a sectioning element that can be displaced by the user in order to be able to select freely the portion to be removed. The corresponding selection by the user is stored in step S2 as a cropping specification information item.

As mentioned above, the specification information established in step S2 is stored and can be used subsequently for a plurality of X-ray images. In step S3, for this purpose, the X-ray image is initially classified into an X-ray image class in order to pick out the correct specification information, whereafter the processing takes place making use of the respective specification information items. Therein, initially a trimming function can be applied which uses the cropping specification information item in order to crop the collimator shadow 18 as needed. Thereafter, the orienting function can be applied which uses the orientation specification information item in order to determine the display orientation, for example as a preliminary orientation and an offset angle, and to orient the X-ray image accordingly in the display orientation. Finally, an enhancement function can be applied which uses the annotation specification information item, in order to enhance the X-ray image already adapted by cropping and rotation, with the respective annotations, in particular at the position provided. In a step S4, the prepared X-ray image is then output on the display facility. As the arrow back to step S3 indicates, the specification information in order to bring about a clinical standardization is directly applied to different X-ray images.

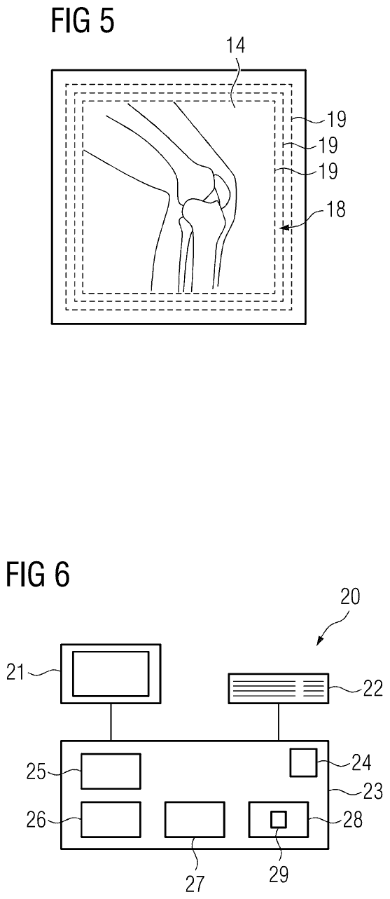

FIG. 6 shows a sketch of the principle of an image processing facility (also referred to as an image processing device) 20 according to one or more example embodiments. It comprises a display facility (also referred to as a display device) 21 and an input facility (also referred to as an input device) 22 which can also be configured partially integrated, for example in the form of a touch screen. The operation of the image processing facility 20 is controlled by a control facility (also referred to as a controller) 23 which has at least one processor (not shown) and a storage means 24 in which the specification information can be stored. The control facility 23 is configured for carrying out the method as described in relation to FIG. 1.

The image processing facility 20 can be or comprise, for example, a diagnostic workstation or can also be or comprise a picture archiving and communication system (PACS). In each case, the control facility 23 can access X-ray images via an interface (not shown in detail) and/or X-ray images can be stored in the storage means 24. The X-ray images can originate from different X-ray facilities, which means that the image processing facility 20 is independent of the scanner.

In the present case, the control facility 23 has a display unit 25 and a user interaction unit 26 with which step S1 of the method can be realized. The display unit 25 also serves to display the processed X-ray image in step S4. In an establishing unit 27, the specification information can be established in accordance with step S2. In a processing unit 28, which in the present case also has an orienting unit 29 as a subunit, the processing of the X-ray image takes place according to step S3. Further subunits of the processing unit 28 can be an enhancing unit and a trimming unit as well as a classification unit.

Independent of the grammatical term usage, individuals with male, female or other gender identities are included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing device) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiments, it is not limited by the disclosed examples and a person skilled in the art can derive other variation herefrom without departing from the protective scope of the invention.

1. A computer-implemented method for operating an image processing device with a display device, an input device and a controller, the method comprising:
for processing an X-ray image for display on the display device, applying at least one orienting function which adjusts a display orientation for the display of the X-ray image to the X-ray image by the controller;
establishing at least one orientation specification information item based on a user input made via the input device; and
selecting the display orientation using the orienting function and the orientation specification information item.

2. The method of claim 1, wherein the establishing establishes the orientation specification information item at least one of for at least one user group as a standard or for a plurality of X-ray image classes, wherein the X-ray image is classified into an X-ray image class before the applying.

3. The method of claim 1, further comprising:
establishing, by the orienting function, a preliminary orientation by evaluating the X-ray image using at least one of (i) a subfunction trained via machine learning, (ii) a guideline information item, (iii) an anatomical landmark in the X-ray image, and
the orientation specification information item comprises an offset angle.

4. The method of claim 3, wherein the input device includes a rotatable operating element configured to adjust the offset angle having a pictogram that shows schematically an X-ray image of an X-ray image class for which the offset angle is to be selected, and a starting orientation of the pictogram is selected according to a predicted orientation without using the orientation specification information item.

5. The method of claim 1, wherein the input device includes a selection operating element, the selection operating element including a selection image which shows schematically anatomical landmarks of an X-ray image of an X-ray image class for which the orientation specification information item is to be selected, and a landmark selected via the selection operating element is stored in the orientation specification information item as specifying the display orientation.

6. The method of claim 1, further comprising:
establishing an annotation specification information item from the user input, wherein for the processing of the X-ray image for display after the applying the orienting function, the X-ray image is annotated in an enhancement function using the annotation specification information item.

7. The method of claim 6, wherein the annotation specification information item relates to an arrangement of an annotation of particular content in the X-ray image.

8. The method of claim 7, wherein for establishing a portion of the user input relating to the annotation specification information item, an input region is displayed on the display device, the input region comprises a schematic map of an X-ray image of an X-ray image class for which the annotation specification information item is to be established, and displaceable annotation elements are displaceable to positions within the map in order to input the portion of the user input.

9. The method of claim 1, further comprising:
establishing a cropping specification information item from the user input, wherein for the processing of the X-ray image for display, the X-ray image is trimmed in a trimming function using the cropping specification information item regarding a collimator shadow in the X-ray image.

10. The method of claim 9, wherein the establishing the cropping specification information item includes offering different removable regions of the collimator shadow for selection via at least one of the input device or a sectioning element that is manipulatable by the user for defining removable regions.

11. An image processing device comprising:
a display device configured to display an X-ray image;
an input device configured to receive a user input; and
a controller configured to cause the image processing device to process an X-ray image for display on the display device, the controller configured to cause the image processing device to,
establish an orientation specification information item from a user input effected via the input device, and
apply to the X-ray image an orienting function which adjusts a display orientation for the X-ray image for display, wherein the display orientation is based on the orienting function using the orientation specification information item.

12. A non-transitory computer readable medium including instructions, when executed on a controller of an image processing device, causes the image processing device to perform the method of claim 1.

13. An non-transitory computer readable medium including instructions, when executed on a controller of an image processing device, causes the image processing device to perform the method of claim 2.

14. The method of claim 2, wherein the orienting function uses an orientation specification information item that associated with the classification.

15. The method of claim 6, wherein the establishing the annotation specification information item establishes the annotation specification information item for each X-ray image class.

16. The method of claim 7, wherein the arrangement of the annotation of particular content in the X-ray image is relative to at least one anatomical landmark.

17. The method of claim 8, wherein the annotation specification information item to be established is in a predicted orientation.

18. The method of claim 9, wherein the establishing establishes the cropping specification information item for each X-ray image class.
